(19) United States
(12) Patent Application Publication
(10) Pub. No.: US 2011/0125225 A1
(43) Pub. Date: May 26, 2011

(54) INTERFERENTIAL NON INVASIV TEMPORARY PACER

(76) Inventor: Narayanan Lakshimanan, Chennai (IN)

(21) Appl. No.: 12/737,470

(22) PCT Filed: Aug. 12, 2008
(86) PCT No.: PCT/IN2008/000508

§ 371 Date: Jan. 18, 2011

(30) Foreign Application Priority Data
Jul. 22, 2008 (IN) 1751/CHE/2008

(51) Int. Cl. A61N 1/36

(52) U.S. Cl. 607/67

(57) ABSTRACT

New effective emergency non invasive pacing technique and apparatus is designed to improve the heart rate, cardiac output in patients during bradycardia. It also overcomes the existing disadvantages of unwanted transcutaneous muscular stimulation in the process of cardiac stimulation. This method uses interferential high frequency currents to selectively produce pulsed stimulus to the desirable area of myocardium is introduced.

### CROSS-REFERENCE TO RELATED APPLICATIONS

This application is a 371 of international application number PCT/IN2008/000508, filed on Aug. 12, 2008.

### REFERENCES CITED

U.S. PATENT DOCUMENTS

| | | |
|---|---|---|
| 5,324,317 | June 1994 | REISS |
| 5,776,173 | July 1998 | MADSEN et al. |
| 2002/0068961 | June 2002 | FISCHER et al. |
| PCT/US2004/001524 | August 2004 | CARROLL |

### BACKGROUND OF THE INVENTION

1. Field of the Invention

This invention relates to the Medical and in particular to non invasive cardiac pacing. In particular it relates to Temporary Emergency Pacing to treat sudden cardiac arrest, to boost low blood pressure due to medical shock and bradycardia non invasively. It also offers solution to existing cutaneous nerve stimulation as well as skeletal muscle stimulation which is associated with the discomfort experienced by the patient in the existing method.

2. Description of the Related Art

The Noninvasive Temporary Pacing (popularly known as NTP) is well known technique used in emergency to manage acute bradycardia to increase the heart rate and cardiac output. Electrical current is passed from an external pulse generator via a conducting cable and externally applied, self adhesive electrodes through chest wall and heart. It is a valuable tool as initial mode of cardiac pacing for brady systolic arrest situations and prophylactic pacing applications. This technique is advantage that any physician can use it with very little training and it is non invasive method and easy of application during emergency.

For emergency pacing modalities, transcutaneous cardiac pacing is a valuable technique as it can be quickly safely and easily initiated by minimally trained personnel. It may be effective when endocardial pacing fails or is contraindicated.

Zoll et. al had proved that stimulating the ventricle would cause it to contract and that such stimulation could be effective through intact chest wall.

Several interferential stimulator devices have previously been described. U.S. Pat. No. 5,776,173 for PROGRAMMABLE INTERFERENTIAL STIMULATOR; issued Jul. 7, 1998 to Madsen teaches an interferential stimulator in general. Such stimulators are useful for stimulating individual nerves or muscles of the extremities. It does not teach about handling cardiac muscles and in particular the whole heart which requires very big stimulating pulse width and high current along with pulsatile stimuli.

Another U.S. Pat. No. 5,324,317 for INTERFERENTIAL STIMULATOR FRO APPLYING LOW FREQUENCY ALTERNATING CURRENT TO THE BODY issued on Jun. 28, 1994 to Reiss teaches an interferential stimulator in general. Such stimulators are useful for stimulating individual nerves or muscles of the extremities. It does not teach about handling cardiac muscles and in particular the whole heart which requires very big stimulating pulse width and high current along with pulsatile stimuli.

Another US Pat Application No 202/68961 for ELECTRICAL STIMUALTOR AND METHOD OF USE applied on Jun. 6, 2002 by Fischer teaches multiple modes of electro-therapy including an interferential stimulator in general. Such stimulators are useful for stimulating individual nerves or muscles of the extremities. It also teaches about galvanic high voltage pulse current mode and does not discuss anything about high voltage pulsed interferential mode. Also it does not teach about handling cardiac muscles and in particular the whole heart which requires very big stimulating pulse width and high current along with pulsatile stimuli.

Another Pat Application No PCT/US2004/001524 for SPINAL CORD STIMULATION WITH INTERFERENTIAL CURRENT applied on Aug. 5, 2004 by Carroll teaches about implantable low current with very small pulse width interferential stimulator. It does not teach about handling cardiac muscles and in particular the whole heart which requires very big stimulating pulse width and high current along with pulsatile stimuli.

Also none of the cited patents teaches the how interferential stimulator can be used as pacemaker. Also none of the cited patents teaches gating of the ECG with the stimulator.

Transcutaneous cardiac pacing produces depolarization of myocardial tissue by pulsed electrical current conducted through the chest between electrodes adherent to the skin. The self-adhesive surface patch electrodes are large in area—typically 8 cm in diameter. Currently available transcutaneous pacing generators may come as stand-alone units or be incorporated in to external defibrillators. Transcutaneous pacing generators should feature asynchronous and demand pacing modes, a built in ECG display that filters the large pacing artifact, widely adjustable settings for pacing rate and current output. Available units provide up to 200 mA of current and use a rectangular or truncated exponential pulse waveform of 20 to 40 msec duration. The long pulse width permit the lowest pacing thresholds while minimizing the stimulation of skeletal muscle and cutaneous nerves. To initiate transcutaneous pacing, the patch electrodes are secured anteriorly and posteriorly to the chest wall.

In emergency bradyasystolic situations or with unconscious patients, transcutaneous pacing should be initiated in the asynchronous mode and at maximal current output to ensure ventricular capture. Chest compressions may be performed directly over the electrodes without disruption of pacing or conduction of significant electrical current to medical personnel. Cardiac capture is suggested by the appearance of depolarization artifacts following the pacing stimuli, but capture must be confirmed by palpation of a pulse or using oximeter. Once capture is documented, the current may be decreased gradually until loss of capture defines the pacing current threshold.

Also, medical shock is a life-threatening medical condition which is very often causes considerable mortality. There are several types of medical shock, septic shock, anaphylactic shock, cardiogenic shock, hypovolemic shock, and neurogenic shock. Causes of shock include heart attack, heart failure, heavy bleeding (internal and external), infection, anaphylaxis, spinal cord injury, severe burns, chronic vomiting or diarrhea. Low blood pressure is the key sign of shock. Treatment is dependant upon the type of shock. Low blood pressure can also be increased by increasing the heart rate and cardiac output. This external non invasive pacing modality will aid such treatment and to sustain life and prevent end organ damage.

Also it is well known that this technique produces painful muscular stimulation, variable incidence of cardiac capture and poor patient tolerance. This discomfort often intolerable restricts external stimulation to use in unconscious patients or to brief use in desperate situations. Because of the pain and supposed ineffectiveness of noninvasive pacing, endocardial pacing was generally preferred even for temporary use, in both emergency and standby situations.

The undesirable side effects of this present technique can be summarized as follows:

1. The current output that produces pain which is highly variable among individuals. Majority of patients cannot be paced a tolerable levels of discomfort.

2. The pain results from simulation of cutaneous afferent nerves and intense pacing induced skeletal muscle contraction.

Also there have been many proposals to overcome this disadvantage like using ramp waveform etc., instead of pulsed current output but these proposals only reduced or likely to reduce the undesirable effects. It cannot overcome the disadvantage of unnecessary pacing induced stimulation of skeletal muscle and cutaneous afferent nerves.

It is therefore an object of invention is to provide a method of stimulating myocardium and or total heart using interferential methodology. Present invention presents a unique approach to the above disadvantage by which it overcomes unnecessary pacing induced stimulation of skeletal muscle and cutaneous afferent nerves. This invention will popularize in emergency settings due to easy of application and minimal training. Also this invention opens up more usage with proper trials only to stimulate a particular area of myocardium.

It is yet further object of invention to provide a traveling wave like stimulus potential which will in particular to heart will be beneficial to increase the cardiac output.

It is yet another object of invention to provide demand mode of stimulation so that intrinsic heart rate can take over the pacing whenever natural rhythm picksup.

### BRIEF SUMMARY OF THE INVENTION

A technique of non invasive cardiac stimulation is disclosed here such that it is very useful in sustaining heart beat and cardiac output in cases of bradycardia, low blood pressure and cardiac arrest. It also overcomes the disadvantage mentioned with non invasive temporary pacer of the current art.

The heart of the invention is that it uses the known phenomena of high frequency currents do not produce neuro-muscular stimulation. It is a known fact that, frequency above 1 KHz do not produce any stimulation. When two such high frequency currents of slightly different frequency, passed intersecting at the area of interest as shown in FIG. (3) then in that focal point (area of intersection) due to interference it produces a pulsating low frequency pulses which will stimulate the focal point. This technique is the basic of heart of invention. This technique produces no stimulation at the current entry point due to high frequency and hence there will be no transcutaneous stimulations at the site of electrodes. Whereas by planning the two circuits of high frequency currents of such a way the intersection of currents exactly located in the ventricular region of myocardium then due to integration of anti-phasic currents produces a perfect stimulus to myocardium in isolation. This technique of stimulating myocardium in isolation without other neuro-muscular system getting stimulated, presents a novel and powerful technique to pace the heart non invasively.

Interferential electrical stimulation's is a unique way of effectively delivering therapeutic stimulus to cardiac tissues. Conventional pacemakers use discrete electrical pulses. However, Interferential pacemaker use a fixed carrier frequency of 4,000 Hz per second in the first channel and a second channel of same frequency but variable phase or pulse width modulated. When the two channel currents combine (heterodyne), they produce the desired signal pulsed beat (Interference beat). Interferential stimulation is concentrated at the point of intersection of currents (between the electrodes). This concentration occurs deep in the tissues as well as at the surface of the skin. Conventional pacemakers deliver most of the stimulation directly under the electrodes. Thus, with Interferential pacemaker, current perfuses to greater depths and over a larger volume of tissue than other forms of electrical therapy. When current is applied to the skin, capacitive skin resistance decreases as pulse frequency increases. For example, at a frequency of 4,000 Hz (Interferential unit) capacitive skin resistance is eighty (80) times lower than with a frequency of 50 Hz. Thus, Interferential current crosses the skin with greater ease and with less stimulation of cutaneous receptors allowing greater patient comfort during electrical stimulation. In addition, because medium-frequency (Interferential) current is tolerated better by the skin, the dosage can be increased, thus improving the ability of the Interferential current to permeate tissues and allowing easier access to deep cardiac structures. This explains why Interferential current is most suitable for treating patients non invasively.

More specifically, the present invention uses two sets of electrodes positioned such that the target area myocardium is located straight intersection of these four electrodes. Diagonally opposite pair of electrodes is connected to one circuit. A isolated high frequency current is passed through the electrode in anti phasic manner.

In accordance with one aspect of invention the base carrier frequency of the current can be anything up to 20 KHz and the channel phase relationship can be varied slightly with reference to one channel to other so that interferential stimulus, stimulates the myocardium.

In accordance with still another aspect of invention, by suitably placing the electrodes, the target stimulus area can be selected such as atrium or ventricle of the heart. This offers the physician better control of pacing and sustain life with better cardiac output.

In accordance with yet another aspect of invention the same pacing electrodes can be used to pick up ECG which will help in avoiding placing separate ECG electrodes for the ECG monitoring. This saves lot of time in cardiac medical emergency.

It will be appreciated that the disclosed method and circuit is advantageous in that it allows only myocardium getting stimulated and hence there is no pain or muscular stimulation and hence well tolerated by the patient. To assist with understanding the invention, reference will now be made to the accompanying drawings, which show one example of invention.

### BRIEF DESCRIPTION OF THE SEVERAL VIEWS OF THE DRAWINGS

The foregoing aspects and many of the attendant advantages of this invention will become more readily appreciated as the same becomes better understood by reference to the detailed description, when taken in conjunction with the accompanying drawings.

FIG. 1 is a prior art of using Non invasive Temporary Pacer showing the pad placement. The FIG. 1a shows Anterior/Lateral electrode placement and 1b shows Anterior/Posterior electrode placement.

FIG. 2. illustrates the principle of Interferential Current.

FIG. 3. illustrates the tissue stimulation using Interferential Current.

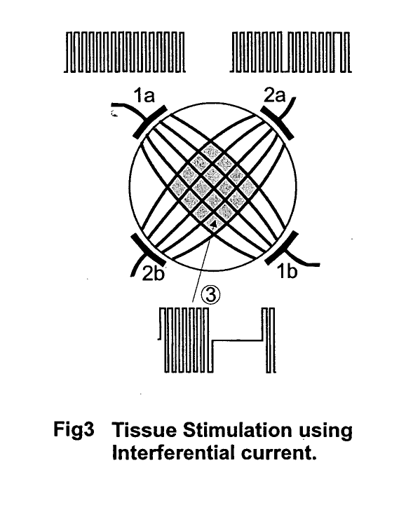

FIG. 4. illustrates the inventive step of passing high frequency current and how the target area getting stimulated.

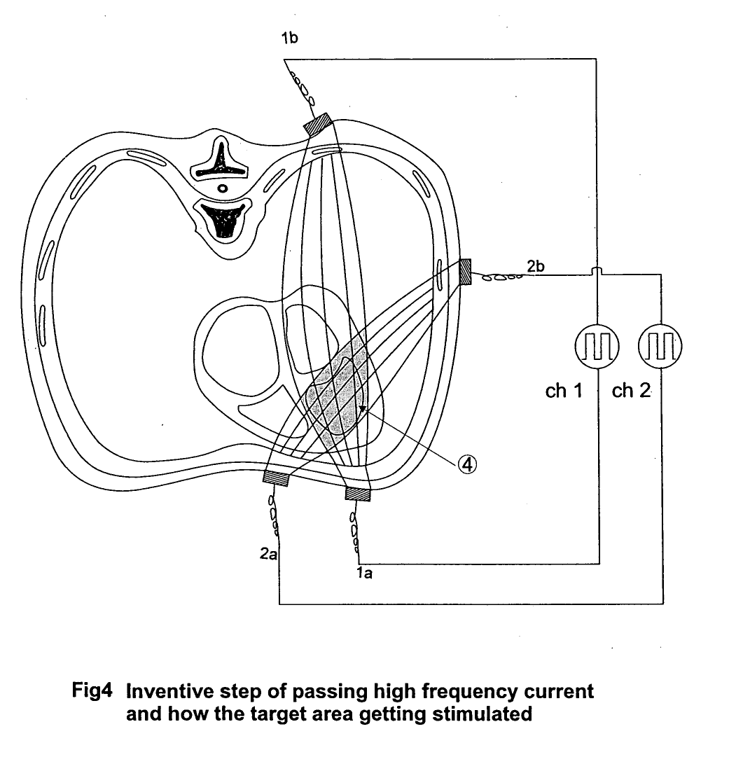

FIG. 5 illustrates the electrode placement position to stimulate different areas of myocardium

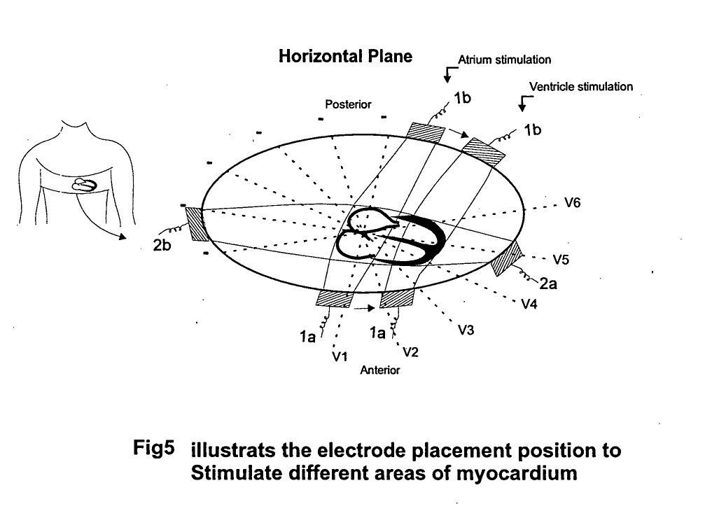

FIG. 6 illustrates method of electrode placement for Interferential Pacing

FIG. 7 illustrates the block diagram of the interferential non invasive pacing.

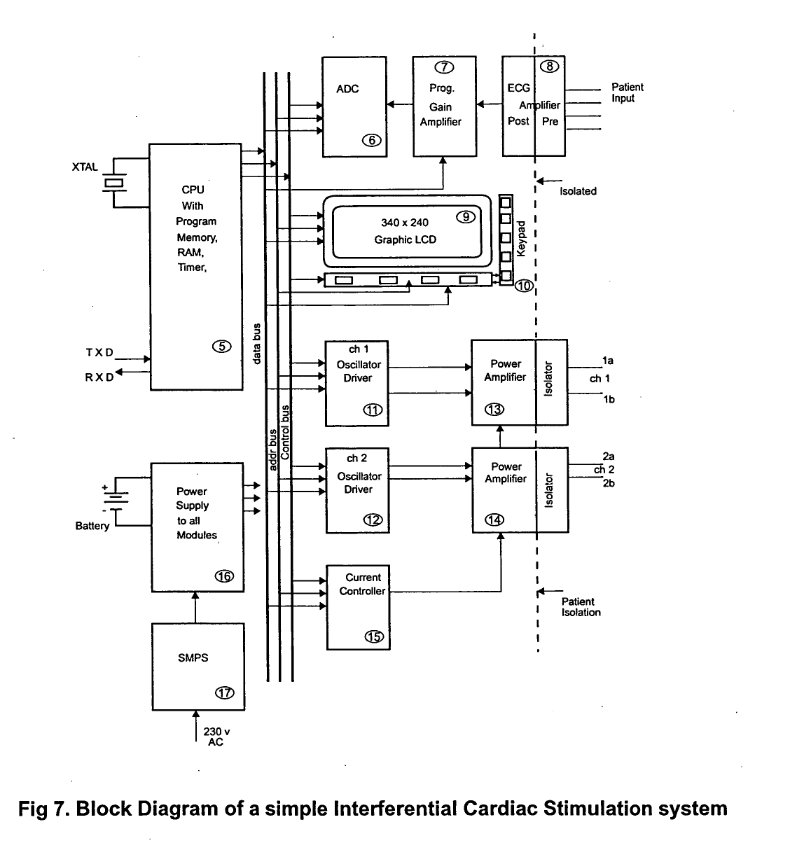

### DETAILED DESCRIPTION OF THE INVENTION

The FIG. (2) to FIG. (6) clearly explains the technique used to stimulate the myocardium the Heart muscle using two high frequency currents. By varying the phase or pulse width modulating one carrier a resultant pulse produced at the target area is one similar to a internal pacemaker waveshape is achieved. This technique of stimulation overcomes all serious disadvantages of prior art. Also user can adjust the stimulation area to get the desired effect.

The general operation of the preferred embodiment of the present invention is illustrated in FIG. (4). This differs from the prior art of NTP that it requires four conducting electrodes along with high frequency currents as shown. The set of electrodes 1a and 1b constitute one circuit and similarly 2a and 2b constitute another isolated circuit. When high frequency currents are passed, the currents heterodyne at the intersection point and resultant beat impulse is produced in the target area (4). FIG. (4) explicitly explains how cardiac stimulation can be achieved by using interferential technique. The location of the pads and its usefulness to select an area of stimulation is described in FIG. (5). By moving set of opposite electrodes 1a & 1b, one can achieve any target area stimulation. May be Atrium or Ventricle can be targeted. This feature will be helpful to improve the cardiac output by an experienced physician by properly stimulating the atrium or ventricle. Placement of electrodes is the key to the selection of area in the myocardium.

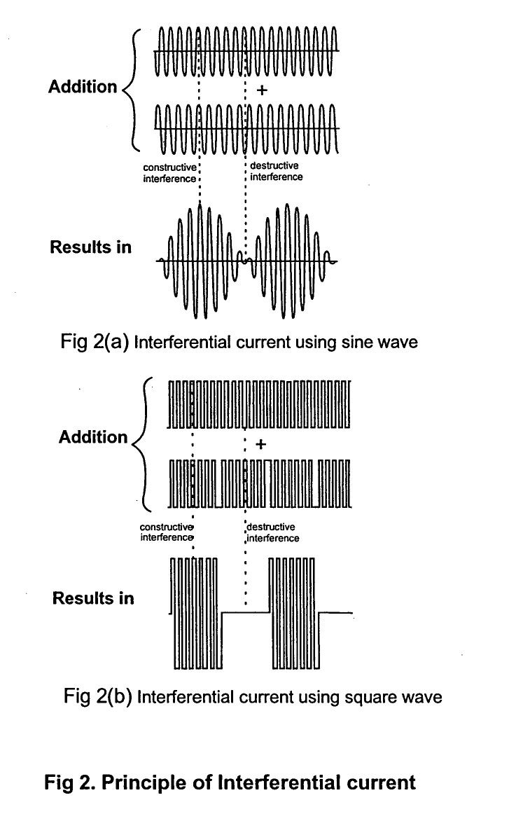

FIGS. (2a) and 2(b) explains the basic working of Interferential currents. FIG. 2(a) illustrates how two channels of Isolated High frequency currents when crosses it heterodyne and produces beating frequency. In FIG. (2b) illustrates how out of phase pulsed high frequency produces the gated pulses. This explains the basic principle behind the myocardium stimulation by using High frequency.

FIG. (3) illustrates how the actual interferential current from two channels from properly positioned electrodes produces stimulation in the intersection path area (3). FIG. (1) illustrates how in prior art the electrodes are positioned.

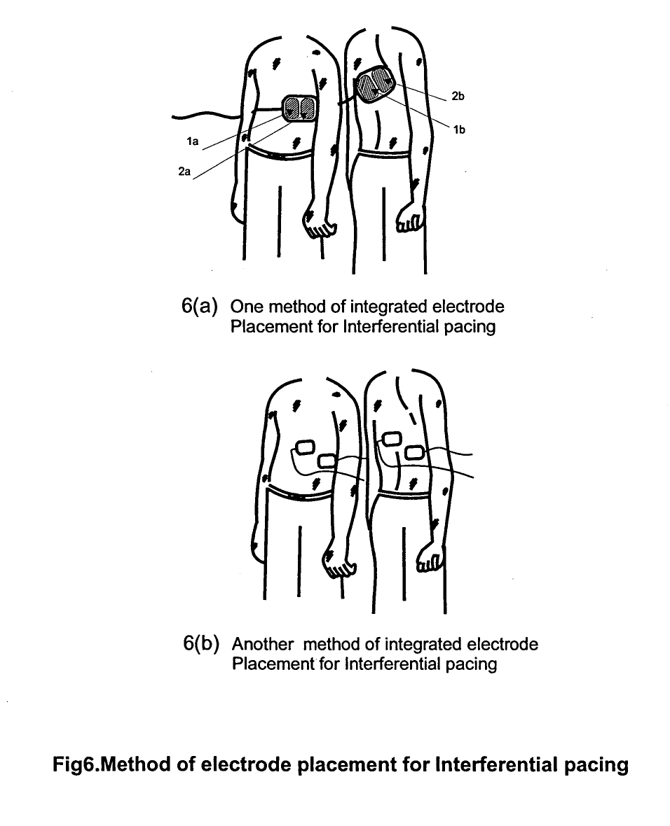

FIG. (6) illustrates the method of electrode placement for interferential pacing. Depending on the area of myocardial stimulation the electrode placements will vary from one end to another in the thorax. There is numerable ways of electrode placement possible to achieve different effects. Meanwhile the FIG. 6(a) illustrates using integrated 2 electrodes like prior art but the one piece of electrode will contain two circuits as shown and with two electrodes two circuits are established to produce interferential currents. This may be easily achievable by using 4 separate electrodes as shown in FIG. 6(b). With FIG. 6 (b) scheme one can adjust and attain stimulation precisely at different parts of myocardium.

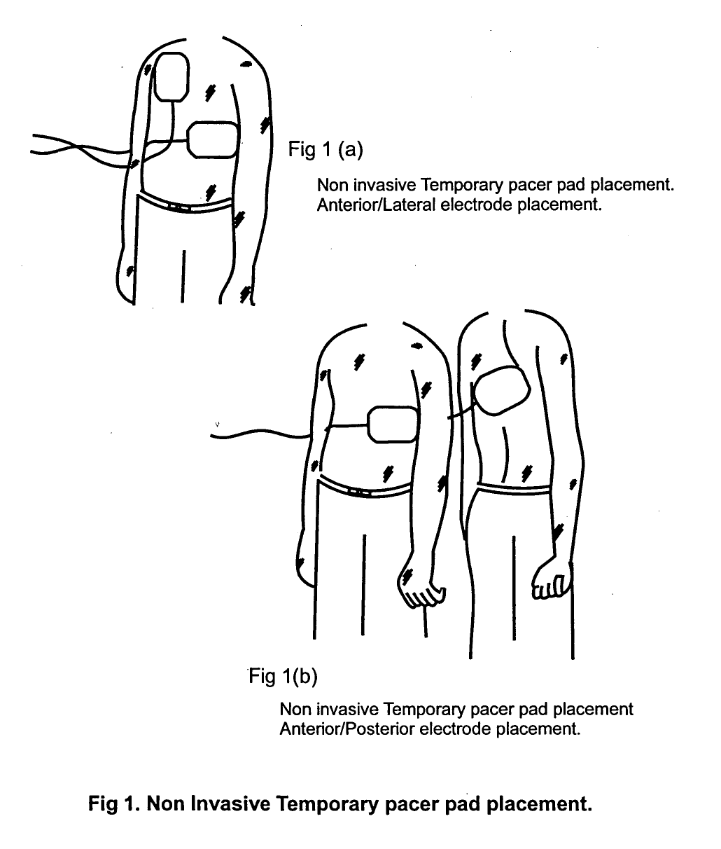

FIG. (1) illustrates the prior art of Non Invasive Temporary pacing using two big electrodes. Two methods of electrode placement is popular as shown in FIG. 1(a) Anterior/Lateral placement and FIG. 1(b) Anterior/Posterior placement.

While the preferred embodiments of the invention has been illustrated and described, it will be apparent that various changes can be made therein without departing from the sprit and scope of the invention. For example, the FIG. (5) electrode positioning is shown in one axis whereas electrode movement can be made in another axis. Also the movement itself be made using an automatic tool. Consequently, within the scope of the appended claims, it will be appreciated that the invention can be practiced otherwise than as specifically described herein.

FIG. (7) illustrates the Block Diagram of Interferential Noninvasive Pacing Device. The CPU (5) is the Master of the Device. It is functionally responsible for the acquisition and processing of ECG, Displaying of ECG, Generating time base for the two channels, controlling the modes of pacing and strength of pacing currents. A microcontroller with inbuilt Program memory, RAM, Timer and I/O Ports is used. The ADC (6) digitize the processed ECG signal fed by Programmable Gain Amplifier (7). The Programmable Gain amplifier is controlled by the CPU for the Gain. The Input ECG amplifier (8) consists of Pre and post amplifiers. The Pre amplifier is fully electrically isolated for the patient safety.

The ECG and Pacing Pulse is displayed as two traces. ECG trace is displayed above and the Pace stimulation pulse is displayed as second trace below. This allows the user to instantly compare the two traces and conclude the cardiac capture due to non invasive stimulation. The display is of Graphic LCD/TFT screen (9) sufficient enough to display two traces. The CPU (5) directly writes in to graphic display and maintains the display content. Also the Keyboard (10) is interfaced to CPU (5). User can program the stimulus frequency, its current strength etc., using this keyboard.

Also the CPU (2) is connected to heart of the Pacer consisting of two channels viz., Channel 1 (11) and Channel 2 (12). Each channel is programmed to base frequency of say 4000 Hz and either one of them can be phase or frequency shifted by the CPU (5) as per the user program programmed earlier. The identical high frequency signals are getting amplified by the respective power amplifiers (13) and (14). The Power amplifier is coupled with Transformer isolator that the output current is fully isolated and independent. The current strength is varied by the CPU (5) directly by using an current controller (15) which biases the Power amplifier to deliver the programmed out put current.

Depending on the setting of the Pace frequency the CPU (5) outputs high frequency pulsed currents via two channels. But the waveforms of the channel are modulated in such a way that the beat frequency process the stimulus pulse at the target area.

In this preferred embodiment it will be appreciated that the both channels are closely controlled by the CPU and the output current is isolated electrically by means of magnetic isolation.

Also it will be appreciated that the Pacer can be put into Asynchronous Mode or in Demand Mode. Again the outputting beat frequency is software controlled accordingly to the mode set by the user.

Power supply section (16) is an DC-DC Converter with battery charger. It supplies all the power requirements of blocks. It also charges the battery whenever the Mains power is connected and present. The Mains Power is converted into DC by SMPS (17).

While the preferred embodiments of the invention has been illustrated and described, it will be apparent that various changes can be made therein without departing from the sprit and scope of the invention. For example, the generation of the bi-phasic high frequency currents can be devised by many means. Consequently, within the scope of the appended claims, it will be appreciated that the invention can be practiced otherwise than as specifically described herein.

What is claimed is:

1-9. (canceled)

10. A method of improving the heart rate, medical shock induced low blood pressure and cardiac output in human beings during bradycardia and thereby sustaining life of a patient non invasively comprising:
Interferential high frequency isolated current passing through means of electrodes attached to the thorax of human body crisscrossing the target area, in particular to a spot or whole area myocardium, heart muscle;
Wherein the said interferential current while crisscrossing the target area produces a pulsed stimulus;
Wherein the said pulsed stimulus stimulates the whole or part of myocardium as selected and the consequently the heart beats;
Wherein the said pulsed stimulus intensity, is selected to achieve the desired cardiac output.

11. A method of controlling an apparatus for treating a patient, external non invasively, the method comprising:
The electrodes are positioned such that the current from the each pair of electrodes crisscrosses the target area, preferably myocardium;
Wherein the electrode position is adjusted so as to get the desired area as target of stimulus;
Wherein the said interferential current, the resultant pulse frequency, namely stimulus per minute, selected by the user;
Wherein the said frequency is selectable from 30 to 250 beats per minute;
Wherein the said interferential current, the intensity is increased from zero to a value manually or automatically, till such a time a cardiac capture is recognized;
Wherein the said interferential current intensity varied from 0 to 250 mA maximum;
Wherein the said interferential current, can be controlled by the apparatus selectively, so as to achieve the demand mode of stimulation, namely when the heart is self sustaining to the required selected frequency.

12. An apparatus for stimulating cardiac muscles by external non invasive technique comprising:
Continuous isolated high frequency current passing through suitable electrodes as base carrier frequency;
Varying the individual channel phase and or width such that pulsed stimulus appears in the target area;
The said continuous isolated high frequency current generated by the generator is of a square or pulse modulated wave with a base frequency of at least 500 Hz but not more than 20 KHZ;
Wherein the said interferential current output waveform include a resultant pulsed waveform with a pulse width with a range of at least 5 msecs but no more than 100 msecs.
Wherein the said interferential current output include a resultant pulsed waveform with pulse repetition frequency of atleast 30 stimulus per minute to 250 stimulus per minute;
Wherein the said interferential current output include a resultant pulsed waveform with an output current to a value no greater than about 250 mA;
Wherein the said interferential current output waveform pulse is biphasic or monophasic or damped sinusoid;
Wherein the said interferential current output is delivered through four independent electrodes, two for one channel placed such a way that current emanating from each set crosses each other to produce the desired effect;
The said electrodes are of at least square cm in size to reduce the skin redness due to I²R losses at the entry point of the skin;
The said generator is controlled by the sensing the patient's ECG (electrocardiogram);

13. An apparatus claimed in claim 12 wherein, interferential high frequency can use either sine wave or square wave or pulse modulated wave or rectangular wave as the base carrier.

14. An apparatus claimed in claim 12 wherein, interferential high frequency current can be either continuous or pulsed.

15. An apparatus claimed in claim 12 wherein, interferential high frequency current is further amplitude modulated to produce a enhanced pulsed stimulating effect in the target area.

16. An apparatus claimed in claim 12 wherein, the electrodes are steered mechanically over the chest to attain the selective stimulation of particular area of heart (myocardium).

17. An apparatus claimed in claim 12 wherein, the electrodes of opposite channel are integrated in to one electrode for easy of application.

18. An apparatus claimed in claim 12 wherein, stimulating cardiac muscles by external non invasive technique comprising:
Multiple continuous isolated high frequency current passing through suitable electrodes as base carrier frequency;
Varying the individual channel phase and or width such that pulsed stimulus appears in the target area;
With multiple channels of generators of more than two, connecting each to two set of electrodes, produces a traveling wave like stimulation in the desired direction in myocardium.

19. An apparatus claimed in claim 12 wherein, sensing the ECG from the pacing electrodes for processing and displaying in the attached cardiac monitor and without use of any further electrodes for monitoring the ECG.
